(19) United States
(12) Patent Application Publication
(10) Pub. No.: US 2010/0251485 A1
(43) Pub. Date: Oct. 7, 2010

(54) DELIVERY PARTICLE

(76) Inventors: Johan Smets, Lubbeek (BE); Susana Fernandez Prieto, Benicarlo (ES)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/731,185

(22) Filed: Mar. 25, 2010

Related U.S. Application Data
(60) Provisional application No. 61/165,941, filed on Apr. 2, 2009.

(51) Int. Cl. A61K 8/11; C11D 3/37; C09K 3/00; D06L 1/00

(52) U.S. Cl. 8/137; 510/515; 252/186.42; 512/4

(57) ABSTRACT

The present application relates to particles, compositions comprising such particles, and processes for making and using such particles and compositions. Such particles minimize or eliminate certain drawbacks of benefit agents including encapsulated benefit agents. When employed in compositions, for example, cleaning or fabric care compositions, such particles increase the efficiency of benefit agent delivery, there by allowing reduced amounts of benefit agents to be employed. In addition to allowing the amount of benefit agent to be reduced, such particles allow a broad range of benefit agents to be employed.

### CROSS-REFERENCE TO RELATED APPLICATIONS

This application claims priority under 35 U.S.C. §119(e) to U.S. Provisional Application Ser. No. 61/165,941, filed Apr. 2, 2009.

### FIELD OF INVENTION

The present application relates to particles, compositions comprising such particles, and processes for making and using such particles and compositions.

### BACKGROUND OF THE INVENTION

Benefit agents, such as perfume delivery compositions, bleaching agents and fabric softening agents, are expensive and generally less effective when employed at high levels in personal care compositions, cleaning compositions, and fabric care compositions. As a result, there is a desire to maximize the effectiveness of such benefit agents. One method of achieving this objective is to improve the delivery efficiencies of such benefit agents. Unfortunately, it is difficult to improve the delivery efficiencies of benefit agents as such agents may be lost due to the agents' physical or chemical characteristics or such agents may be incompatible with other compositional components or the situs that is treated.

In an effort to improve the delivery efficiencies of benefit agents, the industry, in many cases, encapsulated such benefit agents. Unfortunately, in certain applications, a large portion of the resulting capsules rupture prematurely. Thus, there is a need for a particle and/or agglomerate that minimizes or eliminates the aforementioned drawbacks.

### SUMMARY OF THE INVENTION

The present application relates to particles and/or agglomerates, compositions comprising such particles and/or agglomerates, and processes for making and using such particles and/or agglomerates and compositions. Such particles and/or agglomerates minimize or eliminate certain drawbacks of encapsulated benefit agents.

### DETAILED DESCRIPTION OF THE INVENTION

### Definitions

As used herein “consumer product” means baby care, beauty care, fabric & home care, family care, feminine care, health care, snack and/or beverage products or devices intended to be used or consumed in the form in which it is sold, and not intended for subsequent commercial manufacture or modification. Such products include but are not limited to diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, RX pharmaceuticals, pet health and nutrition, and water purification; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); and coffee.

As used herein, the term “cleaning composition” includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, dentifrice, car or carpet shampoos, bathroom cleaners; hair shampoos and hair-rinses; shower gels and foam baths and metal cleaners; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists.

As used herein, the term “fabric care composition” includes, unless otherwise indicated, fabric softening compositions, fabric enhancing compositions, fabric freshening compositions and combinations there of.

As used herein, the phrase “benefit agent delivery particle” encompasses microcapsules including perfume microcapsules.

As used herein, the articles including “a” and “an” when used in a claim, are understood to mean one or more of what is claimed or described.

As used herein, the terms “include”, “includes” and “including” are meant to be non-limiting.

Asked herein, waxes include natural waxes, chemically modified waxes and the synthetic waxes. The natural waxes include, for example, plant waxes such as candelilla wax, carnauba wax, Japan wax, esparto grass wax, cork wax, guaruma wax, rice germ oil wax, sugarcane wax, ouricury wax or montan wax, animal waxes such as beeswax, shellac wax, zein wax, spermaceti, lanolin (wool wax), or uropygial grease, mineral waxes such as ceresin or ozokerite (earth wax), or petrochemical waxes such as petrolatum, paraffin waxes or microwaxes. The chemically modified waxes include, for example, hard waxes such as montan ester waxes, sassol waxes or hydrogenated jojoba waxes. Synthetic waxes include polyalkylene waxes or polyalkylene glycol waxes. Suitable synthetic waxes include higher esters of phthalic acid, in particular dicyclohexyl phthalate, which is obtainable commercially under the name Unimoll® 66 (Bayer AG). Also suitable are synthetic waxes made from lower carboxylic acids and fatty alcohols, for example dimyristyl tartrate which is obtainable under the name Cosmacol® ETLP (Condea). Synthetic or semisynthetic esters of lower alcohols with fatty acids include, for example, Tegin® 90 (Goldschmidt), a glycerol monostearate palmitate.

The test methods disclosed in the Test Methods Section of the present application should be used to determine the respective values of the parameters of Applicants' inventions.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Wax Coated Particles

Applicants discovered that the problem of achieving effective and efficient benefit agent delivery can be solved in an economical manner when the benefit agent and/or melamine formaldehyde encapsulated benefit agent is delivered coated with a wax including but not limited to, shellac and/or zein.

In one aspect, a composition comprising:
1.) a benefit agent selected from the group consisting of:
   a.) an encapsulated benefit agent wherein said encapsulated benefit agent comprises a material selected from the group consisting of a perfume; a perfume delivery composition; a bleaching agent comprising a material selected from the group consisting of a diacyl, a clathrated diacyl, sodium nonanoyloxybenzene sulfonate, a bleach booster, a metal catalyst and mixtures thereof; a fabric softening agent; and mixtures thereof; and a melamine formaldehyde polymer, said melamine formaldehyde polymer encapsulating said material;
   b.) a perfume delivery composition;
   c.) a bleaching agent comprising a material selected from the group consisting of a diacyl, a clathrated diacyl, sodium nonanoyloxybenzene sulfonate, a bleach booster, a metal catalyst and mixtures thereof; and
   d.) a fabric softening agent; and
   e.) mixtures thereof; and
2.) a wax selected from the group consisting of shellac, zein, paraffins and mixtures thereof, said wax covering said benefit agent to form a particle, an agglomerate, or a bead; and
3.) an adjunct material.

In one aspect of the aforementioned composition, whether encapsulated by a melamine formaldehyde polymer or unencapsulated, the diacyl may comprise a material selected from the group consisting of dinonoyl peroxide, didecanoyl peroxide, diundecanoyl peroxide, dilauroyl peroxide, dibenzoyl peroxide, di-(3,5,5-trimethyl hexanoyl) peroxide and mixtures thereof and the aforementioned diacyl component of the clathrated diacyl may be selected from the group consisting of dinonoyl peroxide, didecanoyl peroxide, diundecanoyl peroxide, dilauroyl peroxide, dibenzoyl peroxide, di-(3,5,5-trimethyl hexanoyl) peroxide and mixtures thereof.

In one aspect of the aforementioned composition, whether encapsulated by a melamine formaldehyde polymer or unencapsulated, the perfume delivery composition may comprise a material selected from the group consisting of amine reaction product, a polymer assisted delivery system, starch encapsulated accord, zeolite or inorganic comprising an accord and mixtures thereof. Examples of suitable perfume delivery compositions and processes of making same are found in USPA publications: 2007/0275866 A1; 2008/0200359 A1 and 2008/0200363 A1.

In one aspect of the aforementioned composition, whether encapsulated by a melamine formaldehyde polymer or unencapsulated, the bleach booster may comprise a material selected from the group consisting of:

2-[3-[(2-hexyldodecyl)oxy]-2-(sulfooxy)propyl]-3,4-dihydroisoquinolinium, inner salt;

3,4-dihydro-2-[3-[(2-pentylundecyl)oxy]-2-(sulfooxy)propyl]isoquinolinium, inner salt;

2-[3-[(2-butyldecyl)oxy]-2-(sulfooxy)propyl]-3,4-dihydroisoquinolinium, inner salt;

3,4-dihydro-2-[3-(octadecyloxy)-2-(sulfooxy)propyl]isoquinolinium, inner salt;

2-[3-(hexadecyloxy)-2-(sulfooxy)propyl]-3,4-dihydroisoquinolinium, inner salt;

3,4-dihydro-2-[2-(sulfooxy)-3-(tetradecyloxy)propyl]isoquinolinium, inner salt;

2-[3-(dodecyloxy)-2-(sulfooxy)propyl]-3,4-dihydroisoquinolinium, inner salt;

2-[3-[(3-hexyldecyl)oxy]-2-(sulfooxy)propyl]-3,4-dihydroisoquinolinium, inner salt;

3,4-dihydro-2-[3-[(2-pentylnonyl)oxy]-2-(sulfooxy)propyl]isoquinolinium, inner salt;

3,4-dihydro-2-[3-[(2-propylheptyl)oxy]-2-(sulfooxy)propyl]isoquinolinium, inner salt;

2-[3-[(2-butyloctyl)oxy]-2-(sulfooxy)propyl]-3,4-dihydroisoquinolinium, inner salt;

2-[3-(decyloxy)-2-(sulfooxy)propyl]-3,4-dihydroisoquinolinium, inner salt;

3,4-dihydro-2-[3-(octyloxy)-2-(sulfooxy)propyl]isoquinolinium, inner salt;

2-[3-[(2-ethylhexyl)oxy]-2-(sulfooxy)propyl]-3,4-dihydroisoquinolinium, inner salt; and

mixtures thereof;

In one aspect of the aforementioned composition, whether encapsulated by a melamine formaldehyde polymer or unencapsulated, the metal catalyst may comprise a material selected from the group consisting of dichloro-1,4-diethyl-1,4,8,11-tetraaazabicyclo[6.6.2]hexadecane manganese(II); dichloro-1,4-dimethyl-1,4,8,11-tetraaazabicyclo[6.6.2]hexadecane manganese(II) and mixtures thereof; and mixtures thereof; a fabric softening agent; and mixtures thereof.

In one aspect of the aforementioned composition, such composition may have a weight ratio of benefit agent to wax of from about 95:5 to about 1:99, from about 95:5 to about 15:85, or even from about 90:10 to about 50:50 and a mean particle size of from about 1 micron to about 5 mm, from about 2 microns to about 2 mm or even from about 5 microns to about 100 microns.

In one aspect, of the aforementioned composition, such composition's wax may comprise shellac.

In one aspect, of the aforementioned composition, such composition's wax may encapsulate such composition's benefit agent.

In one aspect, of the aforementioned composition, such composition's wax may encapsulate such composition's benefit agent to form a particle.

In one aspect, of the aforementioned composition, such composition's wax may comprises shellac and such composition's benefit agent may comprise an encapsulated benefit agent that may comprise perfume microcapsules that may comprise perfume and a melamine formaldehyde polymer that may encapsulate said perfume, such particle may have a mean particle size of from about 5 microns to about 100 microns.

In one aspect, of the aforementioned composition, such composition's shellac may comprise borax and/or ammonia.

In one aspect, of the aforementioned composition, such composition's wax may comprise a plasticizer selected from the group consisting of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, hydroxypropyl methylcellulose, benzyl benzoate, butyl and/or glycol esters of fatty acids, refined mineral oils, oleic acid, castor oil, corn oil, camphor, glycerol, sorbic acid, sorbitol and mixtures thereof.

In one aspect, of the aforementioned composition such composition's wax and benefit agent may form an agglomerate particle.

In one aspect, of the aforementioned composition, such composition's benefit agent may comprise a bleaching agent.

In one aspect, of the aforementioned composition, such composition's benefit agent may comprise a material selected from the group consisting of dilauroyl peroxide; dinonoyl peroxide; sodium nonanoyloxybenzene sulfonate; isoquinolinium, 2-[3-[(2-butyloctyl)oxy]-2-(sulfooxy)propyl]-3,4-dihydro-, inner salt; isoquinolinium, 2-[3-[(2-ethylhexyl)oxy]-2-(sulfooxy)propyl]-3,4-dihydro-, inner salt; dichloro-1,4-dimethyl-1,4,8,11-tetraaazabicyclo[6.6.2]hexadecane manganese(II); dichloro-1,4-diethyl-1,4,8,11-tetraaazabicyclo[6.6.2]hexadecane manganese(II); and mixtures thereof.

In one aspect, of the aforementioned composition, such composition's benefit agent may comprise a fabric softening agent.

In one aspect, of the aforementioned composition, such composition's benefit agent may comprise a fabric softening agent selected from the group consisting of a paraffin, an oil, a silicone, a clay and mixtures there of.

In one aspect, a composition that may comprise any combination of the aforementioned parameters as listed in the aforementioned aspects is disclosed.

The suitable materials and equipment for practicing the present invention may be obtained from: United Initiators, GmbH & Co. KG, Dr.-Gustav-Adolph-Str. 3, 82049 Pullach, Germany SSB, Stroever GmbH & Co. KG, Muggenburg 11, 28217 Bremen, Germany; Emerson Resources INC, Suite 1, 600 Markley Street, Norristown, Pa. 19401; Appleton, 825 E Wisconsin Avenue, P.O. Box 359, WI 54912-0359, US; Sigma Aldrich NV/SA, Kardinaal Cardijnplein 8, 2880 Bornem, Belgium; ProCepT nv, Rosteyne 4, 9060 Zelzate, Belgium; Ingeniatrics, Avd. Américo Vespucio 5-4, 1^{a} p., mód. 12, Sevilla, Spain; GEA Process Engineering Inc.•9165 Rumsey Road•Columbia, Md. 21045, US; Mettler-Toledo, Inc., 1900 Polaris Parkway, Columbus, Ohio, 43240, US; IKA-Werke GmbH & Co. KG, Janke & Kunkel Str. 10, 79219 Staufen, Germany; Alfa Aesar GmbH & Co KG, Zeppelinstrasse 7, 76185 Karlsruhe, Germany; Eastman Chemical Company, PO Box 431, Kingsport, Tenn. 37662, US.

### Process of Making

In one aspect, the process of making the aforementioned compositions, including wax coated particles and/or agglomerates may comprise two (2) parts: a) combining and/or contacting a solution comprising a wax, including but not limited to, shellac and/or zein, and a solvent, including but not limited to water and/or ethanol, with melamine-formaldehyde microcapsules comprising a benefit agent and/or a slurry comprising such melamine-formaldehyde microcapsules to form a shellac/microcapsule slurry and b) collecting wax coated melamine formaldehyde microcapsules from such slurry. In one aspect, a wax solution is prepared and a slurry comprising melamine-formaldehyde microcapsules comprising a benefit agent, is added to such solution to form a slurry comprising wax and such melamine-formaldehyde microcapsules. In one aspect, when flow focusing is employed to collect the wax coated melamine formaldehyde microcapsules, the aforementioned slurry is contacted with a second wax solution that may comprise a wax including but not limited to, shellac and/or zein. In one aspect, a plasticizer may be added to the wax/melamine-formaldehyde microcapsule slurry to modify the properties of the resulting wax coated melamine formaldehyde microcapsules—for example to soften the wax coated microcapsules and/or improve the wax coated microcapsules' benefit agent's release during use. Suitable plasticizers include plasticizers selected from the group consisting of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, hydroxypropyl methylcellulose, benzyl benzoate, butyl and/or glycol esters of fatty acids, refined mineral oils, oleic acid, castor oil, corn oil, camphor, glycerol, sorbic acid, sorbitol and mixtures thereof. In one aspect, said plasticizer comprises glycerol. In one aspect, the wax and melamine-formaldehyde microcapsule slurry is combined with an organic material, for example an oil including but not limited to a vegetable oil such as soybean oil, to form a slurry comprising wax, melamine formaldehyde microcapsules and the organic material. Optionally, when the wax and melamine-formaldehyde microcapsule slurry is combined with an organic material, a material that can provide cations may then be combined with the wax, melamine formaldehyde and organic material slurry to assist in hardening the wax coated melamine formaldehyde microcapsules that may be collected from such slurry. In one aspect, the wax and melamine-formaldehyde microcapsule slurry may be contacted with a material that can provide cations—typically such material comprises water and a cation that may be supplied by a salt, such as calcium chloride and/or magnesium and such contact is achieved by passing drops of such slurry through such material that can provide cations. Optionally, when such slurry is contacted with a material that can provide cations, such material may comprise a density modifier such as organic solvent like an alcohol such as ethanol. In one aspect, a second solvent is added to the wax/melamine formaldehyde microcapsule slurry and the first solvent is evaporated which results in wax coated melamine formaldehyde microcapsules in the second solvent. In any of the aforementioned aspects of the invention, the aforementioned slurry may, as needed, be kept homogenous by continual mixing and/or the addition of a surfactant prior to drying. Suitable collecting techniques, include, but are not limited to, spray drying, filtration, flow focusing, and combinations thereof.

In one aspect, a process of making wax coated particles and/or agglomerates may comprise contacting a benefit agent with a fluid wax such as shellac and/or zein, to form wax coated particles. In one aspect said process comprises contacting a benefit agent and/or a melamine formaldehyde benefit agent with a liquid wax such as paraffin, to form wax coated particles. In one aspect, a plasticizer may be combined with the wax to modify the properties of the resulting wax coated particles—for example to soften the wax coated particles and/or improve the wax coated particles' benefit agent's release during use. In one aspect, when flow focusing is employed to collect the wax coated particles, a material selected from the group consisting of a benefit agent, a melamine formaldehyde encapsulated benefit agent, a liquid wax comprising a benefit agent and/or a melamine formaldehyde encapsulated benefit agent and mixtures there of may be contacted with a second wax that may comprise a wax including but not limited to, shellac, paraffin and/or zein. Additional collection techniques include, but are not limited to spray drying, filtration, cooling and combinations thereof.

In one aspect a process of making wax coated particles and/agglomerates may comprise the use of a fluidized bed, wherein a material selected from the group consisting of a benefit agent, a melamine formaldehyde encapsulated benefit agent, a wax coated a benefit agent and/or a wax coated melamine formaldehyde encapsulated benefit agent and mixtures thereof may be contacted with a second wax, that may comprise a wax including but not limited to, shellac, paraffin and/or zein.

### Compositions Comprising Applicants' Compositions

Compositions comprising the aforementioned variants of Applicants' compositions may comprise any embodiment of such variants including the particle variant disclosed in the present application and mixtures of such variants. In one aspect, said composition comprising a variant of Applicants' compositions may be a consumer product. While the precise level of Applicants' composition that is employed depends on the type and end use of the composition, consumer products may comprise, in one aspect, based on total composition weight, from about 0.001% to about 20%, from about 0.001% to about 5%, from about 0.001% to about 1%, from about 0.001% to about any variant or mixture there of Applicants' compositions.

In one aspect, a cleaning composition may comprise, based on total cleaning composition weight, from about 0.1 to about 1 weight % of the Applicants' composition. In one aspect, a fabric treatment composition may comprise, based on total fabric treatment composition weight, from about 0.01 to about 10% of the particles of any variant or mixture there of Applicants' compositions.

Aspects of the invention include the use of the particles of the present invention in laundry detergent compositions (e.g., TIDE™), hard surface cleaners (e.g., MR CLEAN™) automatic dishwashing liquids (e.g., CASCADE™), dishwashing liquids (e.g., DAWN™) Bleach Additives (e.g. Ace) and floor cleaners (e.g., SWIFFER™). The cleaning compositions disclosed herein are typically formulated such that, during use in aqueous cleaning operations, the wash water will have a pH of between about 6.5 and about 12, or between about 7.5 and 10.5. Liquid dishwashing product formulations typically have a pH between about 6.8 and about 9.0. Cleaning products are typically formulated to have a pH of from about 7 to about 12. Techniques for controlling pH at recommended usage levels include the use of buffers, alkalis, acids, etc., and are well known to those skilled in the art.

Fabric treatment compositions disclosed herein typically comprise a fabric softening active (“FSA”). Suitable fabric softening actives, include, but are not limited to, materials selected from the group consisting of quats, amines, fatty esters, sucrose esters, silicones, dispersible polyolefins, clays, polysaccharides, fatty oils, polymer latexes and mixtures thereof.

### Adjunct Materials

While not essential for the purposes of the present invention, the non-limiting list of adjuncts illustrated hereinafter are suitable for use in the instant compositions and may be desirably incorporated in certain embodiments of the invention, for example to assist or enhance performance, for treatment of the substrate to be cleaned, or to modify the aesthetics of the composition as is the case with perfumes, colorants, dyes or the like. It is understood that such adjuncts are in addition to the components that are supplied via Applicants' compositions and other components of products previously disclosed herein. The precise nature of these additional components, and levels of incorporation thereof, will depend on the physical form of the composition and the nature of the operation for which it is to be used. Suitable adjunct materials include, but are not limited to, polymers, for example cationic polymers, surfactants, builders, chelating agents, dye transfer inhibiting agents, dispersants, enzymes, and enzyme stabilizers, catalytic materials, bleach activators, polymeric dispersing agents, clay soil removal/anti-redeposition agents, brighteners, suds suppressors, dyes, additional perfume and perfume delivery systems, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the adjunct ingredients are not essential to Applicants' cleaning and fabric care compositions. Thus, certain embodiments of Applicants' compositions do not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure elasticizing agents, fabric softeners, carriers, hydrotropes, processing aids and/or pigments. However, when one or more adjuncts are present, such one or more adjuncts may be present as detailed below:

Surfactants—The compositions according to the present invention can comprise a surfactant or surfactant system wherein the surfactant can be selected from nonionic and/or anionic and/or cationic surfactants and/or ampholytic and/or zwitterionic and/or semi-polar nonionic surfactants. The surfactant is typically present at a level of from about 0.1%, from about 1%, or even from about 5% by weight of the cleaning compositions to about 99.9%, to about 80%, to about 35%, or even to about 30% by weight of the cleaning compositions.

Builders—The compositions of the present invention can comprise one or more detergent builders or builder systems. When present, the compositions will typically comprise at least about 1% builder, or from about 5% or 10% to about 80%, 50%, or even 30% by weight, of said builder. Builders include, but are not limited to, the alkali metal, ammonium and alkanolammonium salts of polyphosphates, alkali metal silicates, alkaline earth and alkali metal carbonates, aluminosilicate builders polycarboxylate compounds. ether hydroxypolycarboxylates, copolymers of maleic anhydride with ethylene or vinyl methyl ether, 1,3,5-trihydroxybenzene-2,4,6-trisulphonic acid, and carboxymethyl-oxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Chelating Agents—The compositions herein may also optionally contain one or more copper, iron and/or manganese chelating agents. If utilized, chelating agents will generally comprise from about 0.1% by weight of the compositions herein to about 15%, or even from about 3.0% to about 15% by weight of the compositions herein.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in the compositions herein, the dye transfer inhibiting agents are present at levels from about 0.0001%, from about 0.01%, from about 0.05% by weight of the cleaning compositions to about 10%, about 2%, or even about 1% by weight of the cleaning compositions.

Dispersants—The compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The compositions can comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is a cocktail of conventional applicable enzymes like protease, lipase, cutinase and/or cellulase in conjunction with amylase.

Enzyme Stabilizers—Enzymes for use in compositions, for example, detergents can be stabilized by various techniques. The enzymes employed herein can be stabilized by the presence of water-soluble sources of calcium and/or magnesium ions in the finished compositions that provide such ions to the enzymes.

Catalytic Metal Complexes—Applicants' compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methyl-enephosphonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, for example, the manganese-based catalysts disclosed in U.S. Pat. No. 5,576,282.

Cobalt bleach catalysts useful herein are known, and are described, for example, in U.S. Pat. Nos. 5,597,936 and 5,595,967. Such cobalt catalysts are readily prepared by known procedures, such as taught for example in U.S. Pat. Nos. 5,597,936, and 5,595,967.

Compositions herein may also suitably include a transition metal complex of a macropolycyclic rigid ligand—abbreviated as “MRL”. As a practical matter, and not by way of limitation, the compositions and cleaning processes herein can be adjusted to provide on the order of at least one part per hundred million of the benefit agent MRL species in the aqueous washing medium, and may provide from about 0.005 ppm to about 25 ppm, from about 0.05 ppm to about 10 ppm, or even from about 0.1 ppm to about 5 ppm, of the MRL in the wash liquor.

Preferred transition-metals in the instant transition-metal bleach catalyst include manganese, iron and chromium. Preferred MRL's herein are a special type of ultra-rigid ligand that is cross-bridged such as 5,12-diethyl-1,5,8,12-tetraazabicyclo[6.6.2]hexa-decane.

Suitable transition metal MRLs are readily prepared by known procedures, such as taught for example in WO 00/32601, and U.S. Pat. No. 6,225,464.

### Processes of Making and Using Compositions

The compositions of the present invention can be formulated into any suitable form and prepared by any process chosen by the formulator, non-limiting examples of which are described in U.S. Pat. No. 5,879,584; U.S. Pat. No. 5,691,297; U.S. Pat. No. 5,574,005; U.S. Pat. No. 5,569,645; U.S. Pat. No. 5,565,422; U.S. Pat. No. 5,516,448; U.S. Pat. No. 5,489,392; U.S. Pat. No. 5,486,303 all of which are incorporated herein by reference.

### Method of Use and Treated Situs

Any of the compositions and/or products/compositions comprising any aspect of Applicants' compositions disclosed herein may be used to clean or treat a situs inter alia a surface or fabric. Typically at least a portion of the situs is contacted with an embodiment of Applicants' composition and/or /compositions comprising any aspect of Applicants' compositions, in neat form or diluted in a liquor, for example, a wash liquor and then the situs may be optionally washed and/or rinsed. In one aspect, a situs is optionally washed and/or rinsed, contacted with any of the compositions and/or products/compositions comprising any aspect of Applicants' compositions disclosed herein then optionally washed and/or rinsed. For purposes of the present invention, washing includes but is not limited to, scrubbing, and mechanical agitation. The fabric may comprise most any fabric capable of being laundered or treated in normal consumer use conditions. Liquors that may comprise the disclosed compositions may have a pH of from about 3 to about 11.5. Such compositions are typically employed at concentrations of from about 500 ppm to about 15,000 ppm in solution. When the wash solvent is water, the water temperature may range from about 5° C. to about 90° C. and, when the situs comprises a fabric, the water to fabric ratio may be from about 1:1 to about 30:1. In one aspect, a situs that has been treated in accordance with any of the aforementioned methods is disclosed.

### Test Methods

It is understood that the test methods that are disclosed in the Test Methods Section of the present application should be used to determine the respective values of the parameters of Applicants' invention as such invention is described and claimed herein.

(1) Mean Particle Size
The mean particle size of the wax coated particles is determined using a Lasentec M500L-316-K supplied by Mettler-Toledo, Inc., 1900 Polaris Parkway, Columbus, Ohio, 43240, US. The equipment is setup (Lasentec, FBRM Control Interface, version 6.0) as described in the Lasentec manuel, issued February 2000. Software setup and sample analysis is performed using Windox software (Windox XP, version 2002) in the WINDOX manual.

### EXAMPLES

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### Example 1

### 60 wt % Core/40 wt % Wall Shellac Microcapsules in Dry Laundry Composition

A 25% solution of shellac SSB-63-HE-N (SSB, Bremen, Germany) in demi-water is prepared at 60 C and filtered with a 1.2 microns filter (Albet, Dassel, Germany). This solution is cooled to room temperature and 2% Glycerol (Sigma Aldrich) is added as plasticizer. 1200 g of microcapsules (Appleton, Wis., US) containing a perfume composition as benefit agent, are suspended in 1680 g of the previous shellac solution and 500 g demi-water are added, to have a 30% of solids in the suspension. This suspension is stirred for 1 hour at 700 rpm and then introduced in the spray-drier (Niro GmbH, Gemany) using a peristaltic pump (Watson-Marlow, Massachusetts, US). Solid particles are collected and then analyzed by microscopy techniques: SEM (TM-1000, Hitachi), Axio Microscope (Zeiss, Germany) and STEREO microscope (Zeiss, Germany). These particles containing perfume as benefit agent, are mixed in a dry laundry composition as follows,

| | % w/w granular laundry detergent composition | | | | | | |
|---|---|---|---|---|---|---|---|
| Component | A | B | C | D | E | F | G |
| Brightener | 0.1 | 0.1 | 0.1 | 0.2 | 0.1 | 0.2 | 0.1 |
| Soap | 0.6 | 0.6 | 0.6 | 0.6 | 0.6 | 0.6 | 0.6 |
| Ethylenediamine disuccinic acid | 0.1 | 0.1 | 0.1 | 0.1 | 0.1 | 0.1 | 0.1 |
| Acrylate/maleate copolymer | 1.5 | 1.5 | 1.5 | 1.5 | 1.5 | 1.5 | 1.5 |
| Hydroxyethane di(methylene | 0.4 | 0.4 | 0.4 | 0.4 | 0.4 | 0.4 | 0.4 |
| phosphonic acid) | | | | | | | |
| Mono-C₁₂₋₁₄ alkyl, di-methyl, | 0.5 | 0.5 | 0.5 | 0.5 | 0.5 | 0.5 | 0.5 |
| mono-hydroyethyl quaternary | | | | | | | |
| ammonium chloride | | | | | | | |
| Linear alkyl benzene | 0.1 | 0.1 | 0.2 | 0.1 | 0.1 | 0.2 | 0.1 |
| Linear alkyl benzene sulphonate | 10.3 | 10.1 | 19.9 | 14.7 | 10.3 | 17 | 10.5 |
| Magnesium sulphate | 0.4 | 0.4 | 0.4 | 0.4 | 0.4 | 0.4 | 0.4 |
| Sodium carbonate | 19.5 | 19.2 | 10.1 | 18.5 | 29.9 | 10.1 | 16.8 |
| Sodium sulphate | 29.6 | 29.8 | 38.8 | 15.1 | 24.4 | 19.7 | 19.1 |
| Sodium Chloride | 0.1 | 0.1 | 0.1 | 0.1 | 0.1 | 0.1 | 0.1 |
| Zeolite | 9.6 | 9.4 | 8.1 | 18 | 10 | 13.2 | 17.3 |
| Photobleach particle | 0.1 | 0.1 | 0.2 | 0.1 | 0.2 | 0.1 | 0.2 |
| Blue and red carbonate speckles | 1.8 | 1.8 | 1.8 | 1.8 | 1.8 | 1.8 | 1.8 |
| Ethoxylated Alcohol AE7 | 1 | 1 | 1 | 1 | 1 | 1 | 1 |
| Tetraacetyl ethylene diamine | 0.9 | 0.9 | 0.9 | 0.9 | 0.9 | 0.9 | 0.9 |
| agglomerate (92 wt % active) | | | | | | | |
| Citric acid | 1.4 | 1.4 | 1.4 | 1.4 | 1.4 | 1.4 | 1.4 |
| PDMS/clay agglomerates (9.5% wt % | 10.5 | 10.3 | 5 | 15 | 5.1 | 7.3 | 10.2 |
| active PDMS) | | | | | | | |
| Polyethylene oxide | 0.2 | 0.2 | 0.2 | 0.2 | 0.2 | 0.2 | 0.2 |
| Enzymes e.g. Protease (84 mg/g | 0.2 | 0.3 | 0.2 | 0.1 | 0.2 | 0.1 | 0.2 |
| active), Amylase (22 mg/g active) | | | | | | | |
| Suds suppressor agglomerate | 0.2 | 0.2 | 0.2 | 0.2 | 0.2 | 0.2 | 0.2 |
| (12.4 wt % active) | | | | | | | |
| Sodium percarbonate (having | 7.2 | 7.1 | 4.9 | 5.4 | 6.9 | 19.3 | 13.1 |
| from 12% to 15% active AvOx) | | | | | | | |
| Perfume oil | 0.5 | 0.5 | 0.5 | 0.5 | 0.5 | 0.5 | 0.5 |
| Solid perfume particles | 0.4 | 0 | 0.4 | 0.4 | 0.4 | 0.4 | 0.6 |
| Shellac particles* | 1.3 | 2.4 | 1 | 1.3 | 1.3 | 1.3 | 0.7 |
| Misc | 0.1 | 0.1 | 0.1 | 0.1 | 0.1 | 0.1 | 0.1 |
| Balance Water | | | | | | | |

*Shellac particles added as fine powder

and tested in a full washing test using non-coated microcapsules with perfume as benefit agent as reference.

### Example 2

### Beads Generation Entrapping Microcapsules in Shellac Cross-Linked with Calcium by Extrusion

30 g microcapsules slurry with perfume as benefit agent (Appleton, Wis., US) are suspended in 100 g Marcoat 125 solution (Emerson Resources INC, Pennsylvania, US). Using a Nisco encapsulator with a vibration unit nozzle, above suspension is dropped into a 7.5% calcium chloride (Sigma Aldrich) aqueous bath. Particles are kept there for one hour, then aqueous phase is removed and particles are dried 48 hours at room temperature. Particles are suspended in a liquid laundry composition containing hydrogen peroxide for 72 hours at 35 C and then used in a wash with a powder and/or liquid co-detergent to determine microcapsule release. Fabrics are checked with microcopy techniques assessing deposition of microcapsules in dry fabrics and positive odor benefit after rubbing.

### Example 3

### 20 wt % Core/80 wt % Wall Shellac Microcapsules in Liquid Laundry Composition

A 25% solution of shellac SSB-NPU-N (SSB, Bremen, Germany) in demi-water is prepared at 60 C and filtered with a 1.2 microns filter (Albet, Dassel, Germany). This solution is cooled to room temperature and 4% Glycerol (Sigma Aldrich) is added as plasticizer. 400 g of microcapsules (Appleton, Wis., US) containing a perfume composition as benefit agent and containing a 50% of solids, are suspended in 3216 g of the previous shellac solution. This suspension is stirred for 1 hour at 700 rpm and then introduced in the spray-drier (Niro GmbH, Gemany) using a peristaltic pump (Watson-Marlow, Massachusetts, US). Solid particles are collected and then analyzed by microscopy techniques: SEM (TM-1000, Hitachi), Axio Microscope (Zeiss, Germany) and STEREO microscope (Zeiss, Germany). These particles contain perfume as the benefit agent, and they are used in a liquid laundry composition as follows:

| | % w/w liquid laundry detergent | | | |
|---|---|---|---|---|
| | composition | | | |
| Component | A | B | C | D |
| C11.8 linear alkylbenzene | 17.2 | 17.2 | 13.5 | 14.0 |
| sulfonic acid | | | | |
| Neodol 23-5 | | | 5.2 | |
| Neodol 23-9 | 10.4 | 10.4 | 5.2 | 8.4 |
| Citric acid | 5.0 | 5.0 | 4.5 | 4.1 |
| DTPA¹ | 0.3 | 0.3 | 0.2 | 0.2 |
| Ethanolamine | 3.3 | 3.3 | 2.6 | 2.6 |
| Sodium hydroxide | 0.6 | to adjust | to adjust | to adjust |
| | | pH | pH | pH |
| ethoxylated amine polymer | 2.0 | 2.0 | 1.6 | 1.6 |
| ethanol | 2.0 | 2.0 | 2.0 | 2.0 |
| silicone suds suppressor | 0.04 | 0.04 | 0.03 | 0.03 |
| Tinopal CBS-X | 0.2 | 0.2 | 0.2 | 0.2 |
| Perfume | 0.3 | 0.3 | 0.2 | 0.2 |
| Blue EM² | 0.005 | | | |
| Basic Violet 3 (CI 42555)³ | | 0.005 | | |
| Basic Violet 4 (CI 42600)⁴ | | | 0.001 | |
| Acid Blue 7 (CI 42080)⁵ | | 0.0003 | | |
| Thickener | 0.1-0.5 | 0.1-0.5 | 0.1-0.5 | 0.1-0.5 |
| water | balance | balance | balance | balance |
| neat pH (of composition) | 3.2 | 3.2 | 2.5 | 2.7 |
| reserve acidity⁶ | 2.5 | 2.5 | 2.9 | 2.5 |
| Shellac particles⁷ | 1.3 | 2.4 | 1 | 1.3 |
| Misc | 0.1 | 0.1 | 0.1 | 0.1 |
| Balance Water | | | | |

¹diethyleneetriaminepentaacetic acid sodium salt
²polymeric colorant from Milliken
^{3,4}fabric hueing dyes
⁵non-fabric substantive dye
⁶gNaOH/100 g of product
⁷Shellac particles added as fine powder

The liquid laundry detergents of Example 3 are used and tested in a full washing test using free perfume as reference.

### Example 4

20 wt % Core/80 wt % Shellac Coated Dichloro-1,4-diethyl-1,4,8,11-tetraaazabicyclo[6.6.2]hexadecane manganese(II) in liquid laundry composition. A 10% solution of shellac SSB-63-HE-N (SSB, Bremen, Germany) in demi-water is prepared at 60 C and filtered with a 1.2 microns filter (Albet, Dassel, Germany). This solution is cooled to room temperature. 2 g of Dichloro-1,4-diethyl-1,4,8,11-tetraaazabicyclo[6.6.2]hexadecane manganese(II) are added to 98 g of the shellac solution previously prepared and mixed (IKA RW-16-Basic, supplied by IKA-Werke GmbH & Co. KG, Janke & Kunkel Str. 10, 79219 Staufen, Germany) till Dichloro-1,4-diethyl-1,4,8,11-tetraaazabicyclo[6.6.2]hexadecane manganese(II) is completely dissolved. Then a spray-dryer is used to collect the particles (4M8 Spray-Dryer from ProCepT, Belgium). Parameters used in the spray-drying process: nozzle 0.4 mm; schuin 60 cyclone; temperature inlet air 140 C; air flow 0.4 m³/min; feeding speed 2 mL/min with syringe. A Yield of 58.14% is obtained. Particles are collected and then analyzed by SEM (TM-1000, Hitachi) and performance is assessed in a standard laundry washing test.

### Example 5

70 wt % Core/30 wt % Shellac coated sodium nonanoyloxybenzene sulfonate in liquid laundry composition. 400 g of sodium nonanoyloxybenzene sulfonate (Eastman, Tenn., US) are weighed and introduced in a fluid bed coater with wurster (4M8-Fluidbed, ProCepT, Belgium). Hot air is set up at 60 C and shellac Splendid C2 (SSB, Bremen, Germany) is sprayed from the bottom at a rate of 3 mL/min. Material is collected and analyzed by SEM (TM-1000, Hitachi) assessing that coating is not uniform, but standard performance test, after aging the particles in a laundry composition, provides cleaning benefits in laundry.

### Example 6

13 wt % Core/87 wt % Shellac coated sodium nonanoyloxybenzene sulfonate in liquid laundry composition. A 25% solution of shellac SSB-63-HE-N (SSB, Bremen, Germany) in demi-water is prepared at 60 C and filtered with a 1.2 microns filter (Albet, Dassel, Germany). This solution is cooled to room temperature. 13 g of sodium nonanoyloxybenzene sulfonate are added to 348 g of the shellac solution, previously prepared, and mixed (IKA RW-16-Basic, supplied by IKA-Werke GmbH & Co. KG, Janke & Kunkel Str. 10, 79219 Staufen, Germany) till sodium nonanoyloxybenzene sulfonate is completely dissolved. Then a spray-dryer is used to collect the particles (4M8 Spray-Dryer from ProCepT, Belgium). Parameters used in the spray-drying process: nozzle 0.2 mm; temperature inlet air 120 C; air flow 0.4 m³/min; feeding speed 3 mL/min with peristaltic pump. Collected particles have a particle size distribution with a mean of 20.8 microns, analyzed with Lasentec (Mettler-Toledo, Ohio, US) as described above.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
a) a benefit agent selected from the group consisting of:
(i) an encapsulated benefit agent wherein said encapsulated benefit agent comprises a material selected from the group consisting of a perfume; a perfume delivery composition; a bleaching agent comprising a material selected from the group consisting of a diacyl, a clathrated diacyl, sodium nonanoyloxybenzene sulfonate, a bleach booster, a metal catalyst, and mixtures thereof; a fabric softening agent, and mixtures thereof; and
a melamine formaldehyde polymer, said melamine formaldehyde polymer encapsulating said material;
(ii) a perfume delivery composition; a bleaching agent comprising a material selected from the group consisting of a diacyl, a clathrated diacyl, sodium nonanoyloxybenzene sulfonate, a bleach booster, a metal catalyst, and mixtures thereof; a fabric softening agent, and mixtures thereof;
wherein whether encapsulated by a melamine formaldehyde polymer or unencapsulated:
said diacyl comprises a material selected from the group consisting of dinonoyl peroxide, didecanoyl peroxide, diundecanoyl peroxide, dilauroyl peroxide, dibenzoyl peroxide, di-(3,5,5-trimethyl hexanoyl) peroxide and mixtures thereof;
said clathrated diacyl comprises a material selected from the group consisting of dinonoyl peroxide, didecanoyl peroxide, diundecanoyl peroxide, dilauroyl peroxide, dibenzoyl peroxide, di-(3,5,5-trimethyl hexanoyl) peroxide and mixtures thereof;
said bleach booster comprises a material selected from the group consisting of: 2-[3-[(2-hexyldodecyl)oxy]-2-(sulfooxy)propyl]-3,4-dihydroisoquinolinium, inner salt; 3,4-dihydro-2-[3-[(2-pentylundecyl)oxy]-2-(sulfooxy)propyl]isoquinolinium, inner salt; 2-[3-[(2-butyldecyl)oxy]-2-(sulfooxy)propyl]-3,4-dihydroisoquinolinium, inner salt; 3,4-dihydro-2-[3-(octadecyloxy)-2-(sulfooxy)propyl]isoquinolinium, inner salt; 2-[3-(hexadecyloxy)-2-(sulfooxy)propyl]-3,4-dihydroisoquinolinium, inner salt; 3,4-dihydro-2-[2-(sulfooxy)-3-(tetradecyloxy)propyl]isoquinolinium, inner salt; 2-[3-(dodecyloxy)-2-(sulfooxy)propyl]-3,4-dihydroisoquinolinium, inner salt; 2-[3-[(3-hexyldecyl)oxy]-2-(sulfooxy)propyl]-3,4-dihydroisoquinolinium, inner salt; 3,4-dihydro-2-[3-[(2-pentylnonyl)oxy]-2-(sulfooxy)propyl]isoquinolinium, inner salt; 3,4-dihydro-2-[3-[(2-propylheptyl)oxy]-2-(sulfooxy)propyl]isoquinolinium, inner salt; 2-[3-[(2-butyloctyl)oxy]-2-(sulfooxy)propyl]-3,4-dihydroisoquinolinium, inner salt; 2-[3-(decyloxy)-2-(sulfooxy)propyl]-3,4-dihydroisoquinolinium, inner salt; 3,4-dihydro-2-[3-(octyloxy)-2-(sulfooxy)propyl]isoquinolinium, inner salt; 2-[3-[(2-ethylhexyl)oxy]-2-(sulfooxy)propyl]-3,4-dihydroisoquinolinium, inner salt; and mixtures thereof;
said metal catalyst comprises a material selected from the group consisting of dichloro-1,4-diethyl-1,4,8,11-tetraaazabicyclo[6.6.2]hexadecane manganese(II); dichloro-1,4-dimethyl-1,4,8,11-tetraaazabicyclo[6.6.2]hexadecane manganese(II) and mixtures thereof; and;
b) a wax selected from the group consisting of shellac, zein, paraffins and mixtures thereof, said wax covering said benefit agent to form a particle, an agglomerate, or a bead; and
c) an adjunct material.

2. The composition of claim 1, wherein, whether encapsulated by a melamine formaldehyde polymer or unencapsulated, said perfume delivery composition comprises a material selected from the group consisting of amine reaction product, a polymer assisted delivery system, starch encapsulated accord, inorganic comprising an accord and mixtures thereof.

3. The composition of claim 1, said composition having a weight ratio of benefit agent to wax of from about 95:5 to about 1:99, from about 95:5 to about 15:85, or even from about 90:10 to about 50:50 and a mean particle size of from about 1 micron to about 5 mm, from about 2 microns to about 2 mm or even from about 5 microns to about 100 microns.

4. The composition of claim 1, said composition having a weight ratio of benefit agent to wax of from about 95:5 to about 1:99, from about 95:5 to about 15:85, or even from about 90:10 to about 50:50 and a mean particle size of from about 1 micron to about 5 mm, from about 2 microns to about 2 mm or even from about 5 microns to about 100 microns.

5. The composition of claim 1, said composition having a weight ratio of benefit agent to wax of from about 95:5 to about 1:99, from about 95:5 to about 15:85, or even from about 90:10 to about 50:50 and a mean particle size of from about 1 micron to about 5 mm, from about 2 microns to about 2 mm or even from about 5 microns to about 100 microns.

6. The composition of claim 3, wherein said wax comprises shellac.

7. The composition of claim 6, said wax encapsulates said benefit agent to form a particle.

8. The composition of claim 7, wherein said wax comprises shellac and said benefit agent comprises an encapsulated benefit agent comprising perfume microcapsules comprising perfume and a melamine formaldehyde polymer encapsulating said perfume, said particle having a mean particle size of from about 5 microns to about 100 microns.

9. The composition of claim 7, wherein said shellac comprises borax and/or ammonia.

10. The composition of claim 8, wherein said wax comprises a plasticizer selected from the group consisting of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, hydroxypropyl methylcellulose, benzyl benzoate, butyl and/or glycol esters of fatty acids, refined mineral oils, oleic acid, castor oil, corn oil, camphor, glycerol, sorbic acid, sorbitol and mixtures thereof.

11. The composition of claim 8 wherein said wax and said benefit agent forms an agglomerate particle.

12. The particle of claim 11 wherein said benefit agent comprises a bleaching agent.

13. The particle of claim 12 where said bleaching agent comprises dilauroyl peroxide; dinonoyl peroxide; sodium nonanoyloxybenzene sulfonate; isoquinolinium, 2-[3-[(2-butyloctyl)oxy]-2-(sulfooxy)propyl]-3,4-dihydro-, inner salt; isoquinolinium, 2-[3-[(2-ethylhexyl)oxy]-2-(sulfooxy)propyl]-3,4-dihydro-, inner salt; dichloro-1,4-dimethyl-1,4,8,11-tetraaazabicyclo[6.6.2]hexadecane manganese(II); dichloro-1,4-diethyl-1,4,8,11-tetraaazabicyclo[6.6.2]hexadecane manganese(II); and mixtures thereof.

14. The particle of claim 11 wherein said benefit agent comprises a fabric softening agent.

15. The particle of claim 14 wherein said fabric softening agent is selected from the group consisting of a paraffin, an oil, a silicone, a clay and mixtures there of.

16. A consumer product comprising the particle according to claim 1.

17. A method of treating and/or cleaning a situs, said method comprising
a.) optionally washing and/or rinsing said situs;
b.) contacting said situs with a particle according to any one of claims 1 and/or a consumer product according to claim 16; and
c.) optionally washing and/or rinsing said situs.

18. A situs treated with the particle of anyone of claims 1 and/or the consumer product of claim 16.
